(19) United States
(12) Patent Application Publication
(10) Pub. No.: US 2021/0364492 A1
(43) Pub. Date: Nov. 25, 2021

(54) METHOD OF CLASSIFYING A PLASTIC

(71) Applicant: ENGEL AUSTRIA GmbH, Schwertberg (AT)

(72) Inventors: Ruth MARKUT-KOHL, Vienna (AT); Klaus FELLNER, Steyr (AT); Thomas KOEPPLMAYR, Linz (AT); Guenther KLAMMER, Aschbach Markt (AT)

(21) Appl. No.: 16/880,062

(22) Filed: May 21, 2020

(51) Int. Cl. G01N 33/44; C08J 5/00; G01K 1/02; G01J 5/00; G01F 22/00; G01L 5/00; G01P 3/44; B29C 45/70; B29C 45/78

(52) U.S. Cl. CPC G01N 33/442; C08J 5/00; G01K 1/026; G01J 5/0037; G01F 22/00; G01L 5/00; B29K 2077/00; B29C 45/70; B29C 45/78; C08J 2377/02; C08J 2323/06; C08J 2323/12; G01P 3/44

(57) ABSTRACT

A method of classifying a plastic, wherein the plastic is plasticized by supplying plasticizing energy in the form of mechanical and/or thermal energy with an increase in a temperature of the plastic from an initial temperature value to a final temperature value, a volume and/or a mass of the plastic and the supplied plasticizing energy is detected as measurement parameters by measurement means, in dependence on the detected measurement parameters, the initial temperature value, and the final temperature value at least one of the following is ascertained: a thermal capacity of the plastic and/or a change in enthalpy of the plastic and/or a parameter which can be derived by calculation from the thermal capacity and/or the change in enthalpy, and a plastic group including the plastic is identified on the basis of the ascertained thermal capacity and/or the ascertained change in enthalpy and/or the parameter which can be derived therefrom by calculation.

The present invention concerns methods of classifying a plastic.

Hereinafter, the state of the art is described by means of shaping machines, with the example of an injection molding machine being discussed in particular. Besides injection molding machines, the class of shaping machines, however, also includes injection presses, presses and the like. The description of the state of the art hereinafter applies to them accordingly.

When setting up an injection molding process, basic settings are in practice generally still made by an operator of the injection molding machine. This also applies to settings of the plasticizing process, by means of which a granular plastic material is converted into a shapeable material, for example using a plasticizing screw

In that respect, the operator is restricted to his experience and to a ‘trial and error’ procedure because the non-linear nature of the plasticizing and shaping process largely hindered an automatic setting of the injection molding machine in the state of the art, although there have long been efforts to develop injection molding machines with the capacity of automatic (self)-setting. Because of the material dependency of the process and its above-mentioned non-linear nature, an automatic setting of the injection molding machine often fails due to the fact that the plastic used is not known or the operator inputs in that respect cannot be checked by an automated procedure.

The object of the present invention is to provide a method by means of which a plastic can be classified and which can be carried out at reasonable effort on or at least in the immediate proximity of a shaping machine.

In a first implementation of the invention this object is attained by the features of claim 1. That is effected in that:
a) the plastic is plasticized by supplying plasticizing energy in the form of mechanical and/or thermal energy with an increase in a temperature of the plastic from an initial temperature value to a final temperature value,
b) a volume and/or a mass of the plastic as well as the supplied plasticizing energy are detected as measurement parameters by measurement technology,
c) in dependence on the detected measurement parameters, the initial temperature value, and the final temperature value at least one of the following is ascertained: a thermal capacity of the plastic and/or a change in enthalpy of the plastic and/or a parameter which can be derived by calculation from the thermal capacity and/or the change in enthalpy, and
d) a plastic group including the plastic is identified on the basis of the ascertained thermal capacity and/or the ascertained change in enthalpy and/or the parameter which can be derived therefrom by calculation.

That is to say, the plastic is classified by way of determining the thermal capacity and/or the change in enthalpy. By virtue of the fact that the kind of plastic and therefore the properties thereof can be ascertained by the method according to the invention—preferably in an automated fashion—achieving an automatic setting of a shaping machine is a considerable step closer, as many of the settings regarding shaping machines, of course, crucially depend on the material being used.

In a second implementation of the invention this is effected by the features of claim 20, more specifically in that:
a) the plastic is plasticized by supplying plasticizing energy in the form of mechanical and/or thermal energy with an increase in a temperature of the plastic from an initial temperature value to a final temperature value,
e) an infrared radiation emitted by the plastic is detected by measurement technology, and
f) a plastic group containing the plastic is identified on the basis of the detected infrared radiation.

In a third implementation of the method the object is attained by the features of claim 22. That is effected in that:

a) the plastic is plasticized by supplying plasticizing energy in the form of mechanical and/or thermal energy with an increase in a temperature of the plastic from an initial temperature value to a final temperature value, and
g) identification information about the plastic is obtained from at least two different sources with at least one of the at least two different sources being a measurement at an apparatus used for plasticizing the plastic, and that on the basis of the identification information a plastic group containing the plastic is identified and preferably the plastic is identified.

What those implementations of the invention have in common is that a plastic group containing the plastic or even the plastic itself is identified on the basis of the detected information, more specifically on an apparatus for plasticizing the plastic, that is to say, during the plasticizing operation. An example of such an apparatus would. of course, be a plasticizing assembly, as for example is used with an injection molding machine, or more generally a shaping machine.

An indication according to which a plastic or a plastic group is identified on the basis of a parameter or another item of information (for example features d), f) or g)) does not mean in the context of the invention that only the given parameter or the other information is used to identify the plastic or the plastic group. Rather, it is always possible to use further parameters, measurement values or additional information for the identification. The Identification of the plastic or the plastic group can also refer to mixtures or two or more plastics or mixtures of plastics with fillers.

The invention offers further advantages. By way of example, the invention makes it possible to estimate the loading on the shaping machine that occurs over time, or to provide a further checking device, by means of which the operation and operational reliability can be monitored.

The plastic groups which are used as classification for the plastic employed include, for example, polyethylenes, polypropylenes, polystyrenes, polyamides, polycarbonates, polymethylmethacrylates and the like.

Besides identification of the plastic group including the plastic, the plastic can also be ascertained per se, that is to say, it is possible to ascertain which plastic is especially involved (for example, PE 0.95, if necessary, even with statement of origin (PE X from the manufacturer Y)). In accordance with the invention, the step of determining the plastic itself is also considered as identification of the plastic group including the plastic. The term ‘determining the plastic’ can also be interpreted in such way that, in addition to classification of the plastic per se, fillers (for example fibres) are identified or the amount thereof in the plastic is ascertained.

A further advantage of the invention is that the method according to the invention can be implemented in an automated fashion so that a plasticizing assembly and/or a shaping machine automatically determines the plastic being used.

Besides the method according to the invention, protection is also claimed for
a shaping machine with the plastic which is plasticized and classified with the method according to the invention, in particular in an injection molding process, being used,
a plasticizing assembly for carrying out a method according to the invention, as well as
a shaping machine which is suitable in particular for carrying out a method according to the invention with a plasticizing assembly according to the invention.

In the figurative sense, by determining in accordance with the invention the thermal capacity and/or the change in enthalpy, a plasticizing assembly according to the invention and/or a shaping machine according to the invention can be used as a calorimeter.

The use of the expression ‘final temperature value’ does not mean that no further change in temperature—in particular an increase in temperature—of the plastic is effected or can occur. The expression merely means that this is the temperature substantially at the end of that period of time over which the respective operation of determining the thermal capacity and/or the change in enthalpy is carried out. Thermodynamic processes following the same can, of course, be carried out and are even inevitable in certain situations. The same applies for the initial temperature value.

The detected measurement parameters (volume and/or mass, plasticizing energy) can be measured directly or indirectly. This, of course, also applies to the initial and final temperature values. The parameters derived by calculation from the thermal capacity and/or the change in enthalpy can equally either be measured directly or indirectly (that is to say, as an independent physical or chemical parameter) or the thermal capacity and/or the change in enthalpy can be measured and the parameters derived by calculation can be calculated from those measurement values. The description hereinafter in part abstains from explicitly indicating the parameter which is derived by calculation from the thermal capacity and/or the change in enthalpy. It is, however, always covered by the definition when talking about (determining) the thermal capacity and/or the change in enthalpy.

Detection by measurement techniques of the infrared radiation emitted by the plastic (method step e)) can preferably be performed at the beginning of the plasticizing operation according to method step a) if, for example, the plastic is still in granulate form. It is, however, also possible for the infrared radiation to be detected by measurement techniques during or after the plasticizing operation.

Further advantageous embodiments of the invention are defined in the appendant claims.

It can preferably be provided that an energy balance and/or a power balance for the plastic is set up on the basis of the detected measurement parameters and the thermal capacity, the change in enthalpy and/or the parameter which can be derived therefrom by calculation is determined in dependence on the energy balance and/or the power balance.

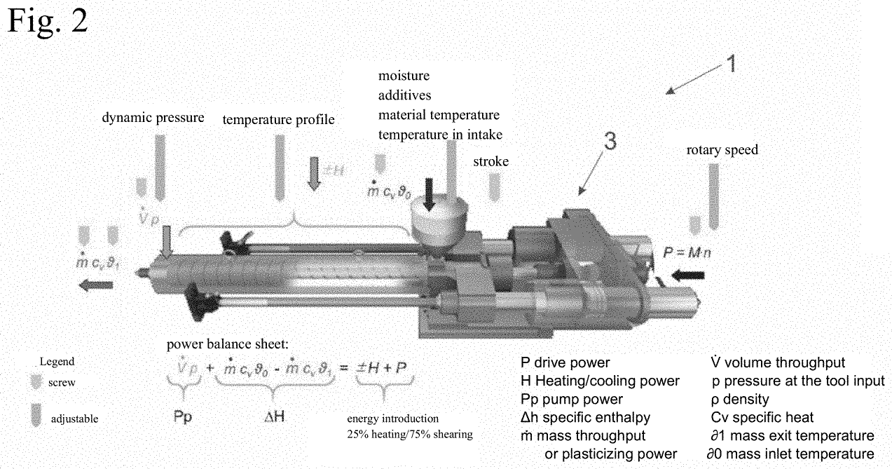

In embodiments which are particularly preferred in that respect, the various contributions to the energy balance and/or the power balance are either known to a certain degree of accuracy, or they can be ascertained at reasonable effort (of course, with the exception of the thermal capacity and/or change in enthalpy to be determined). This way the energy balance and/or the power balance can serve as a basis for determining the thermal capacity and/or the change in enthalpy (as an example for that see FIG. 2).

For that purpose, for example, the energy balance and/or power balance can be resolved by simple mathematical operations according to the parameters being sought (thermal capacity, change in enthalpy). In principle, of course, other operations could also be used to determine the thermal capacity and/or change in enthalpy from the energy balance and/or the power balance (for example, functional minimization/variation calculation).

Establishing an energy or power balance which at least to some degree reflects reality can dramatically increase the accuracy of the thermal capacity and/or change in enthalpy found—and therefore, of course, also the quality of classification of the plastic. Alternatives, however, in principle involve various more or less precise approximations or estimations.

It has already been mentioned that on the basis of the energy balance or the power balance it is possible to ascertain further parameters, more specifically, for example, a load spectrum, a wear state and/or an operating state. A load spectrum concerns a measurement in respect of the total loading over a period of time, preferably over the entire previous service life of the installation. Therefore, the load spectrum can be regarded as a direct measurement in terms of installation wear. Of course, other, in particular more specific wear measures can be determined from the data determined according to the invention, for example, regarding a plasticizing screw or a plasticizing cylinder. Also the operating state of the installation can be quantified by the data ascertained.

The thermal energy and/or thermal power supplied and/or discharged (for example by means of a heating device) and/or a progression of the supplied thermal energy and/or thermal power can be ascertained and taken into consideration for the energy balance and/or power balance. This way the energy or power balance can be particularly easily established.

Besides supplying thermal energy in the plasticizing process regarding plastics, in some cases it is also necessary to cool the plastic, for example, to prevent the temperature of the plastic from going so high that degradation—in the sense of unwanted chemical or physical changes at excessively high temperatures—of the plastic occurs.

Ascertaining the thermal capacity can be particularly easy if it is determined as a specific thermal capacity at a constant pressure and if the plastic group including the plastic is identified on the basis of the specific thermal capacity at a constant pressure. Alternatively, the thermal capacity can be determined at a constant volume or in hybrid form, which, however, gives rise to challenges in the form of in part complex calculations regarding data evaluation and operating states which are difficult to control.

The initial temperature value and/or the final temperature value and/or a temperature progression of the plastic can be measured by means of at least one temperature sensor directly and/or indirectly, wherein preferably temperature regulation of the plastic is performed with the supplied mechanical energy and/or the supplied thermal energy as a setting parameter and measurement values of the at least one temperature sensor as actual values. Using temperature sensors allows for particularly simple and reliable temperature establishment, wherein both direct measurement of the temperature of the plasticized plastic or the plastic not yet plasticized can be implemented and indirect measurement by way of determining the temperature of a body which is in thermal contact with the plastic. In case only a temperature progression is measured, the initial and/or final temperature value can, of course, be found in the progression.

The plastic can be plasticized at a substantially constant pressure, wherein the pressure is preferably closed-loop controlled with a constant target value using measurement values of at least one pressure sensor. The advantages in carrying out the method at constant pressure have already been referred to. Regulating the (mass) pressure to a constant target value is anyway effected in many cases regarding plasticizing assemblies of the state of the art so that the method according to the invention is easy to implement.

The thermal energy can be supplied by means of a—preferably electrical—heating apparatus, which is a particularly simple implementation of the invention, wherein a thermal heating energy and/or a heating power and/or a progression in the heating energy and/or the heating power generated by the heating apparatus can be detected.

It is possible to use at least one drive sensor, by means of which the mechanical energy supplied by way of at least one drive and/or a mechanical power delivered by at least one drive (directly or indirectly on the plastic) and/or a progression in the supplied mechanical energy and/or the delivered mechanical energy is measured. This also supports an easy set-up of an energy or power balance. Regarding the energy or power balance, losses, which, for example, occur due to friction and other resistances, can be taken into account by correction factors—for example, depending on the machine size and efficiency.

It applies to the heating power or energy as well as to the supplied mechanical energy that measured progressions or progressions detected otherwise can be used in order to find the parameters that are actually wanted or to calculate them from the progression (for example, by way of integration).

In a particularly simple implementation of the invention a plasticizing screw and/or a plasticizing piston can be used for supplying the mechanical energy. Plasticizing screws in particular represent a wide-spread structure for plasticizing assemblies, whereby in some cases the invention can be put into effect without structural modifications to the installation. As a rule, in this case there can be provided a plasticizing cylinder in which the plasticizing screw is arranged, wherein the plasticizing screw for plasticizing the plastic is rotationally and axially moved by means of the at least one drive in the plasticizing cylinder. The plastic is sheared by the rotation of the plasticizing screw, wherein at the same time a pressure in the plastic—which, as already mentioned, is preferably controlled to be constant —. is maintained by the axial drive During this so-called dosing operation the screw is moved axially back under said maintaining of the pressure.

A pressure sensor can, however, also directly detect the pressure in the plastic, for example, in the form of a mass pressure sensor in the plasticizing cylinder.

It can be provided that a torque exerted on the plasticizing screw and/or a rotary speed of the plasticizing screw is measured to detect the supplied mechanical energy.

Both the rotation as well as the axial movement can be produced by way of one or more electric or hydraulic drives. Torque and axial movement as well as the application of an axial force can be measured by way of suitable sensors. For example, the torque exerted can be determined from currents and voltages applied to an electric machine driving the rotary movement. The axial force exerted by a hydraulic drive can, for example, be detected by way of the pressure of a hydraulic fluid.

It is possible to use a screw position sensor, by means of which the volume of the plastic plasticized in the plasticizing cylinder is detected. By virtue of the known (cylindrical) geometry it is possible from the screw position to detect the volume—for example before and after plasticizing the plastic—in the plasticizing cylinder

A hopper can be used for supplying the plastic to be plasticized to the plasticizing cylinder, wherein preferably a hopper temperature sensor is used in and/or at the hopper. The initial temperature value of the plastic can be easily ascertained by way of such a hopper temperature sensor without having to put in significant efforts regarding operating methods or structure for that purpose.

In theory, it is also possible to fit to the hopper a weighing device by means of which the mass of the plastic to be plasticized can be measured.

A plurality of cylinder temperature sensors can be used, that are associated with heating zones axially distributed on the plasticizing cylinder, wherein the heating zones are heated independently from each other, wherein the heating in the heating zones is closed-loop controlled respectively using measurement values of the cylinder temperature sensors associated with the heating zones. Such closed-loop controlled heating zones are, too, already present in many existing plasticizing assemblies, and that simplifies the implantation of the invention. Similar of the mechanical supply of energy, losses can also occur here which can be taken into account by correction factors—for example depending on the machine size and the efficiency thereof. Ultimately, that generally applies to all those losses that occur when supplying energy (or also when discharging energy).

Those closed-loop controlled heating zones can be particularly advantageously used for ascertaining the final temperature value by shutting down the closed-loop control of a given heating zone—in particular the last one in the direction of conveying movement of the plastic—and by using a measurement value of the cylinder temperature sensor associated with the given heating zone as the final temperature value. In that case, where possible it can be waited for some time until it is to be expected that the given heating zone is in a state of thermal equilibrium so that the plasticizing cylinder in the region of the given heating zone assumes the temperature of the plasticized plastic. This way, the final temperature value can be measured with a high level of accuracy but without using a mass temperature sensor which in general is relatively expensive. The level of accuracy can be even more increased if the plasticizing assembly is operated in the extrusion mode—that is to say, with a continuous discharge of the plastic from the plasticizing cylinder, as is conducive to a thermally stable state.

The above-mentioned alternative of a dedicated mass temperature sensor which measures the temperature of the plasticized plastic directly, of course, arises nonetheless.

It can further be provided that for identifying the plastic group including the plastic:
enthalpy temperature curves for a plurality of plastics are corrected by means of relationships known for said plastics between the temperature on the one hand and the specific volume and/or the density on the other hand,
the thermal capacity of the plastic and/or the change in enthalpy of the plastic and/or the parameter which can be derived by calculation from the thermal capacity and/or the change in enthalpy are matched to the corrected enthalpy temperature curves—in particular gradients of the corrected enthalpy temperature curves—, and
the plastic group containing the plastic is identified on the basis of the matching.

Particularly the thermal capacity can take over the role of a curve gradient in (corrected) enthalpy temperature curves, whereby matching by calculation or graphical means of the curves with the given values for the thermal capacity and/or the change in enthalpy is particularly easy to perform.

In those cases in which the density or the mass of the plastic to be plasticized is difficult to achieve, this configuration of the method according to the invention allows reliable determination of the plastic or the plastic group containing the plastic.

The method according to the invention can be carried out several times with preferably varying a pressure and/or a supplied mechanical power. The latter, for example, can be varied by a variation in the speed of screw rotation. That affords a particularly simple way of varying the test conditions without having to implement far-reaching changes in the closed-loop control of the heating means (in particular the heating zones).

A near infrared sensor (NIR sensor) can be used for detecting the infrared radiation. In principle, it is also possible to use a (NIR) spectrometer.

Information about the plastic can be obtained from at least one of the following sources and used for identification of the plastic group and preferably the plastic:
methods according to the first implementation of the invention with determination of a thermal capacity of the plastic and/or a change in enthalpy of the plastic and/or a parameter which can be derived by calculation from the thermal capacity and/or the change in enthalpy,
methods according to the second implementation of the invention with detection by measurement techniques of the infrared radiation emitted by the plastic,
machine settings and/or measurement values and/or a machine configuration, in particular injection pressures (or parameters derived therefrom like viscosity or flow rates), maximum and/or minimum processing temperatures, mass cylinder temperatures at the tip or over the entire length of the plasticizing cylinder, granulate temperatures, cooling fluid temperatures, a density of the melt and information about a mold tool used,
methods for determining a parameter characteristic of a compression behavior of the plastic, in particular a compression module or a compressibility, and
camera imaging of the plastic to be plasticized, in particular plastic granulate.

Parameters characteristic of the compression behavior of a plastic like the compression module or compressibility can, for example, be determined in accordance with a method as disclosed in DE 102016005780 A1. The operation of determining amounts of a filler by way of determining compressibility is disclosed in DE 102007030637 A1.

Regarding machine settings and/or measurement values and/or machine configurations, certain plastic groups, for example, have quite particular minimum or maximum processing temperatures. If such particular processing temperatures occur, then the plastic group or the plastic can already be determined or at least restricted thereby. Data of further components or external devices can also be used for identification of the plastic or the plastic group. Relevant data can, for example, occur in driers, material conveyors and/or storage means. Each plastic group has a given characteristic in regard to all above-mentioned parameters. This way, together with the invention according to the first implementation (determining a thermal capacity of the plastic and/or a change in enthalpy of the plastic and/or a parameter which can be derived by calculation from the thermal capacity and/or the change in enthalpy) and/or the second implementation (detection by measurement of the infrared radiation emitted by the plastic) and/or a parameter characteristic of a compression behavior of the plastic the plastic group involved can therefore be ascertained in accordance with an exclusion method and in many cases the plastic can be identified together with loading (fillers).

For setting up the camera image of the plastic to be plasticized, in particular granular plastic material, a camera arranged in or on a hopper of a plasticizing unit can be provided in order, for example, to recognize the granulate form of the plastic granular material.

Determining the granulate form (or other form like powder, for example) can be done by means of an image recognition software—preferably automatically. This way, for example rod-shaped pellets (long glass fibers) can be distinguished from conventional granulate (for example, cylindrical cold pelleting, like typically in the case of technical plastics, or for example, round or lenticular granulate occurring in underwater granulation, typically in the case of polyolefins) or powder (for example PVC Dryblend).

For performing method steps regarding the detection and/or calculation of parameters, and identifying the plastic or the plastic group containing the plastic, the plasticizing assembly and/or the shaping machine can include an evaluation apparatus.

The evaluating apparatus can be adapted to initiate specific steps on the basis of the identified plastic group, for example:
to output indications in respect of improved machine settings and/or improved machine configurations—preferably, after previous automatic identification of one of a current machine settings and/or installed and/or used components—, and/or
to automatically alter machine settings, and/or
to output warnings and/or indications in respect of inadmissible operating states occurring and/or to be expected, and/or
upon inadmissible operating states to output shut-down signals for an automatic shut-down.

The evaluation unit can be integrated in a central machine control of the shaping machine. Alternatively, the evaluation unit can be arranged remotely from the shaping machine and/or the plasticizing assembly and connected thereto, for example, by way of a remote data transmission connection (key word: cloud computing).

The method according to the invention can be performed both in the context of a reference cycle as well as during production. If the plastic group is already known before the beginning of production, correct automatic setting of the shaping process can be ensured. However, even during operation the method according to the invention can be advantageously used in order, for example, to check that the material being processed (the plastic) has not changed.

The reference cycle can be carried out before the beginning of production with a certain plastic to be identified. For that purpose, specific settings can be made on the machine in order to be able to carry out the measurements under conditions which are defined in relation to certain aspects.

Further advantages and details of the invention will be apparent from the Figures and the related specific description. In the Figures:

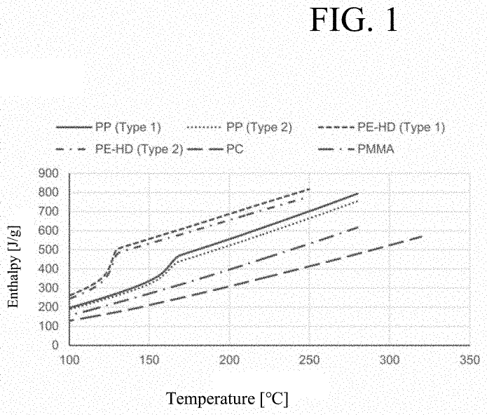

FIG. 1 shows enthalpy temperature curves for different plastics,

FIG. 2 shows a view of a plasticizing assembly with an illustration of a power balance according to the invention,

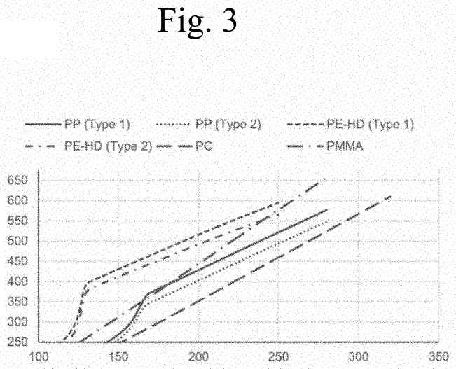

FIG. 3 shows a comparison of the enthalpy temperature curves with a given thermal capacity,

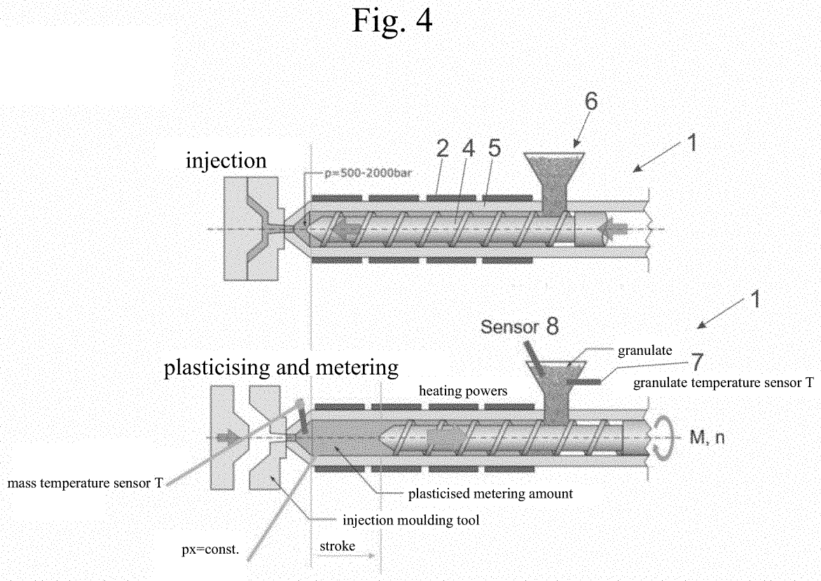

FIG. 4 shows two diagrams to illustrate the plasticizing/metering operation, and

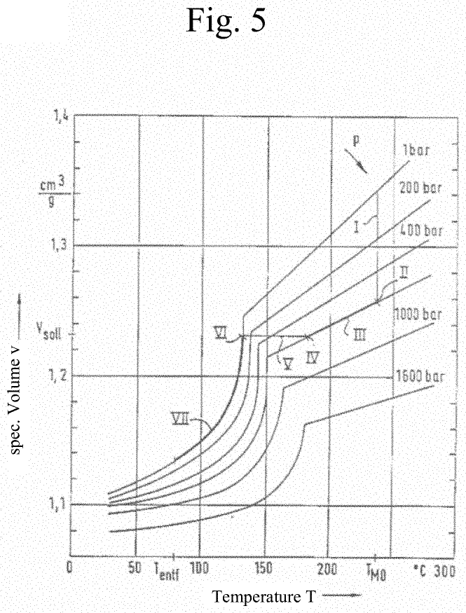

FIG. 5 shows an example of a known temperature dependency of the specific volume for a given plastic.

With a given configuration of a plasticizing assembly, on the one hand, the service life of the installed components is to be optimized while, on the other hand, the processing parameters are to be autonomously adapted in regard to the requirements concerning the plastic to be processed or they are to be recommended.

In order to use optimization algorithms, knowledge of the plastics/plastic groups being processed is absolutely required or at least of substantial use.

A further benefit of the invention according to the first implementation lies in the calculation of a load spectrum: It is possible to calculate characteristic values which provide information about the appropriate use. Those values can also be used as a calculation basis for a leasing business model.

The enthalpy (H) diagram in FIG. 1 known from the literature and the power balance of the system of the plasticizing unit (FIG. 2) serve as the basis for the ascertainment procedure.

The diagram in FIG. 1 describes the energy content/the delivered power of the respective plastic in dependence on the mass temperature T (temperature of the plastic). H=f(T) applies in relation to a kilogram of plastic, the unit is J/g. In addition, the specific volume v with the unit cm³/g has a physically known relationship to the mass temperature T in ° C. and pressure p as shown in FIG. 5.

If the values of the functions H are divided point-wise by the values of the specific volume v at the same temperature with the proviso of a constant pressure, then that relationship gives a function H(pₓ)=f(T) with the unit J/cm³. That function is shown in FIG. 3.

That functional relationship is prepared for selected plastic groups using data technology and is available for controlling the machine.

In the metering operation in an injection molding process the plasticizing screw 4 is rotated at a speed n. In that situation the torque M is provided by the drive 3. At the same time, the screw is withdrawn, thereby setting a melt cushion in the screw pre-chamber. The withdrawal speed in that case is closed-loop controlled in such way that the pressure in the screw pre-chamber pₓ remains constant (see FIG. 4).

The power introduced mechanically into the plasticizing process is proportional to the product of the torque M and the rotary speed n. That can be ascertained on the injection molding machine by virtue of evaluation of the electrical or hydraulic drive parameters.

In relation to FIG. 2 it should also be mentioned that, for example, the following parameters which occur in the plasticizing operation can be set from the exterior: pressure pₓ (dynamic pressure), temperature profile (predetermined for open-loop or closed-loop control of the heating apparatuses), moisture content at the hopper 6, additives in the plastic (that is to say, for example, in the plastic granulate), temperature of the material, temperature in the hopper, rotary speed of the screw. The remaining parameters (for example, torque, stroke, mass flow) are then set during the plasticizing operation.

FIG. 4 shows a plasticizing assembly 1, namely in the state during injection (above) and in the state during or after the metering operation (below). Granulate, for example, is fed to the plasticizing cylinder 5 by way of a hopper 6 and plasticized by means of the plasticizing screw 4. Provided on the plasticizing cylinder 5 is a heating apparatus 2, by means of which the plasticizing cylinder 5 is heated zone-wise. Additional temperature sensors can also be provided (not shown) for zone-wise closed-loop control of the heating power. In regard to the arrangement of the drive 3 of the plasticizing screw 4, reference is to be made to FIG. 2. A hopper temperature sensor 7 and a near infrared sensor 8 are provided on the hopper 6.

In addition, the heating power Q is taken into account in the power balance of FIG. 2. The proportion of the heating power is also ascertained and added to the mechanical power input. In order to be able to ascertain the increase in enthalpy, the power introduced is reduced by the pressure build-up power. That is calculated from the product of the volume flow V ascertained in relation to the stroke movement and the pressure px which is kept constant over the screw stroke movement. The power input into the plastic, ascertained in that way, can now be compared to the curve families available in the control system. Now, if the mass temperature T is known from a measurement on the machine, it is possible to calculate an intersection of the energy content with the mass temperature in FIG. 3, said intersection is also brought into conformity with a curve from the data set. That curve is assigned to a plastic or a plastic group. Therefore, the requirements for a plastic-specific mode of operation or monitoring are provided.

In relation to the third implementation of the invention four embodiments by way of example are also set forth hereinafter. It is generally the case that the plastics or plastic groups are identified in accordance with the invention by way of a logical combination of the per se known chemical and physical properties.
1. Besides 30% glass fibers the plastic sought, also contains ‘carbon black’ as a black coloring agent. Determining the chemical structure of the plastic by means of an NIR sensor is not unambiguous, as the fillers (in particular graphite) absorb the radiation in the near infrared range and the plastic thereby becomes invisible for that characterization method. Some plastics can be excluded by virtue of further process parameters like the processing or drying temperature. Common plastics like polyolefins (polyethylene, polypropylene) are not pre-dried and are processed at below 260° C. Other plastics like polyamides are pre-dried at at least 80° C. and processed at over 260° C. With characterization methods like energy absorption (enthalpy) or compression capability (bulk modulus) it is possible to deduce a given filler content from calibration curves or the plastic group can be further restricted.
2. The plastic being sought contains a specific structure of the molecular chains like, for example, with PA6 or PA66. With PA66 the carbonamide groups are always opposite each other in such way that each functional group can form a hydrogen bridge without deformation of the molecules. With PA6, however, that is possible only at every second carbonamide group. The higher melting point of PA66 and the lower degree of water absorption can be explained by the different molecular structure. By means of a conventional NIR sensor which is calibrated to individual wavelengths the distinction between PA6 and PA66 is not unambiguous and it is necessary to revert either to an NIR spectrometer or another characterization method. Evaluation of process data like the processing temperature and energy absorption in dependence on temperature on the basis of calibration curves can be used here for distinction purposes.
3. The plastic being sought has a known chemical structure and a specific density like, for example, PE-HD (high density) and PE-LD (low density). The basic structure can be recognized by means of an NIR sensor. Evaluation of process data like the processing temperature and the energy absorption in dependence on temperature on the basis of calibration curves as well as characterization methods like determining the compression modulus can be used here in addition.
4. The plastic being sought has a known chemical structure and a specific viscosity, like for example, a polypropylene with a melt mass flow rate MFR=5 g/10 min and a different polypropylene with 50 g/10 min (measured in accordance with DIN EN ISO 1133). The basic structure can be recognized by means of an NIR sensor. For determining the viscosity differences more precisely it is possible to use characterization methods like the flow number which is ascertained from the injection pressure progression. In addition, it is possible to deduce a plastic group in a specific viscosity range from the ratio of energy absorption by dissipation (power requirement of the metering drive) and energy absorption by thermal conduction (power requirement of external heating/cooling).

1. A method of classifying a plastic, wherein
a) the plastic is plasticized by supplying plasticizing energy in the form of mechanical and/or thermal energy with an increase in a temperature of the plastic from an initial temperature value to a final temperature value,
b) a volume and/or a mass of the plastic and the supplied plasticizing energy is detected as measurement parameters by measurement means,
c) in dependence on the detected measurement parameters, the initial temperature value, and the final temperature value at least one of the following is ascertained:
a thermal capacity of the plastic and/or a change in enthalpy of the plastic and/or a parameter which can be derived by calculation from the thermal capacity and/or the change in enthalpy, and
d) a plastic group including the plastic is identified on the basis of the ascertained thermal capacity and/or the ascertained change in enthalpy and/or the parameter which can be derived therefrom by calculation.

2. The method according to claim 1, wherein an energy balance and/or a power balance for the plastic is set up on the basis of the detected measurement parameters and the thermal capacity, the change in enthalpy and/or the parameter which can be derived therefrom by calculation is determined in dependence on the energy balance and/or the power balance.

3. The method according to claim 2, wherein at least one of the following is ascertained on the basis of the energy balance and/or the power balance: load spectrum, wear state, operating state.

4. The method according to claim 2, wherein supplied and/or discharged thermal energy and/or thermal power and/or a progression in the supplied thermal energy and/or thermal power is ascertained and is taken into account for the energy balance and/or power balance.

5. The method according to claim 1, wherein a specific thermal capacity at a constant pressure is determined as the thermal capacity and the plastic group including the plastic is identified on the basis of the specific thermal capacity at a constant pressure.

6. The method according to claim 1, wherein the initial temperature value and/or the final temperature value and/or a temperature progression of the plastic is measured by means of at least one temperature sensor directly and/or indirectly, wherein preferably closed-loop temperature control of the plastic is performed with the supplied mechanical energy and/or the supplied thermal energy as a setting parameter and measurement values of the at least one temperature sensor as actual values.

7. The method according to claim 1, wherein the plastic is plasticized at a substantially constant pressure, wherein the pressure is preferably closed-loop controlled with a constant target value using measurement values of at least one pressure sensor.

8. The method according to claim 1, wherein the thermal energy is supplied by means of a—preferably electrical—heating apparatus.

9. The method according to claim 1, wherein at least one drive sensor is used by means of which the mechanical energy supplied by way of at least one drive and/or a mechanical power delivered by the at least one drive and/or a progression in the supplied mechanical energy and/or the delivered mechanical power is measured.

10. The method according to claim 1, wherein a plasticizing screw and/or a plasticizing piston is used for supplying the mechanical energy.

11. The method according to claim 9, wherein a plasticizing cylinder in which the plasticizing screw is arranged is used and that the plasticizing screw for plasticizing the plastic is moved rotationally and axially by means of the at least one drive in the plasticizing cylinder.

12. The method according to claim 11, wherein a torque exerted on the plasticizing screw and/or a rotary speed of the plasticizing screw is measured for detecting the supplied mechanical energy.

13. The method according to claim 6, wherein the pressure of the plastic is closed-loop controlled as a setting parameter by means of a force exerted on the plastic by the plasticizing screw.

14. The method according to claim 10, wherein a screw position sensor is used by means of which the volume of the plastic plasticized in the plasticizing cylinder is detected.

15. The method according to claim 5, wherein a hopper is used for feeding the plastic to be plasticized into the plasticizing cylinder, wherein preferably a hopper temperature sensor is used in and/or at the hopper.

16. The method according to claim 6, wherein a plurality of cylinder temperature sensors are used that are associated with heating zones axially distributed on the plasticizing cylinder with the heating zones being heated independently of each other, wherein heating in the heating zones is closed-loop controlled respectively using measurement values of the cylinder temperature sensors associated with the heating zones.

17. The method according to claim 16, wherein in order to ascertain the final temperature value, the closed-loop control of a given heating zone—in particular the last one in the direction of conveying movement of the plastic—is shut down and a measurement value of the temperature sensor associated with the given heating zone is used as the final temperature value.

18. The method according to claim 1, wherein to identify the plastic group including the plastic:
enthalpy temperature curves for a plurality of plastics are corrected by means of relationships known for said plastics between the temperature on the one hand and the specific volume and/or the density on the other hand,
the plurality of values for the thermal capacity of the plastic and/or the change in enthalpy of the plastic and/or the parameter which can be derived by calculation from the thermal capacity and/or the change in enthalpy are matched to the corrected enthalpy temperature curves—in particular gradients of the corrected enthalpy temperature curves —, and
the plastic group containing the plastic is identified on the basis of the matching.

19. The method according to claim 1, wherein the method is carried out several times with preferably a pressure and/or a supplied mechanical power being varied.

20. A method of classifying a plastic, wherein
b) the plastic is plasticized by supplying plasticizing energy in the form of mechanical and/or thermal energy with an increase in a temperature of the plastic from an initial temperature value to a final temperature value,
h) an infrared radiation emitted by the plastic is detected by measurement means, and
i) a plastic group containing the plastic is identified on the basis of the detected infrared radiation.

21. The method according to claim 20, wherein a near infrared sensor is used for detecting the infrared radiation.

22. A method of classifying a plastic, wherein
b) the plastic is plasticized by supplying plasticizing energy in the form of mechanical and/or thermal energy with an increase in a temperature of the plastic from an initial temperature value to a final temperature value, and
j) identification information about the plastic is obtained from at least two different sources, wherein at least one of the at least two different sources is measurement at an apparatus used for plasticizing the plastic and that on the basis of the identification information a plastic group containing the plastic is identified and preferably the plastic is identified.

23. The method according to claim 22, wherein information about the plastic is obtained from at least one of the following values and used for identifying the plastic group and preferably the plastic:
detected infrared radiation or identification information about the plastic,
machine settings and/or measurement values and/or a machine configuration, in particular injection pressures (or parameters derived therefrom like viscosity or flow rates), maximum and/or minimum processing temperatures, mass cylinder temperatures at the tip or over the entire length of the plasticizing cylinder, granulate temperatures, cooling fluid temperatures, a density of the melt and information about a mold tool used,
methods of determining a parameter characteristic of a compression behavior of the plastic, in particular a compression module or a compressibility, and
camera imaging of the plastic to be plasticized, in particular plastic granulate.

24. A shaping method, wherein the plastic which is plasticized and classified with the method according to claim 1, is used in particular during an injection molding method.

25. A plasticizing assembly adapted to carry out the method according to claim 1.

26. The plasticizing assembly according to claim 25, wherein the plasticizing assembly is adapted on the basis of the identified plastic or identified plastic group:
to output indications in relation to improved machine settings and/or improved machine configurations—preferably after previous automatic identification of a current machine setting and/or installed and/or used components —, and/or
to automatically alter machine settings, and/or
to output warnings and/or indications in relation to inadmissible operating states occurring and/or to be expected, and/or
upon inadmissible operating states to output shut-down signals for automatic shut-down.

27. A shaping machine, in particular for carrying out the method according to claim 24, comprising a plasticizing assembly.

28. The shaping machine according to claim 27, wherein the evaluation unit is integrated in a central machine control system of the shaping machine.

29. The shaping machine according to claim 27 for carrying out a shaping method, wherein the shaping machine is adapted to carry out the shaping method in the course of a reference cycle and/or during production.
